(19) United States
(12) Patent Application Publication
(10) Pub. No.: US 2004/0034405 A1
(43) Pub. Date: Feb. 19, 2004

(54) Axially expanding polymer stent

(76) Inventor: Andrew M. Dickson, Gallatin, TN (US)

Correspondence Address: Woodard, Emhardt, Naughton, Moriarty And McNett, Bank One Center/ Tower, Suite 3700, 111 Monument Circle, Indianapolis, IN, 46204-5137, US

(21) Appl. No.: 10/205,932

(22) Filed: Jul. 26, 2002

(51) Int. Cl. A61F 2/06

(52) U.S. Cl. 623/1.11

(57) ABSTRACT

This invention relates to an deformable stent or catheter formed of a shape-memory polymeric material (SMP). The stent is originally molded into a desired configuration for treatment of a variety of medical treatments. Subsequently, the stent can be deformed to a second configuration selected to facilitate implantation. Either during implantation or after implantation, the stent can be stimulated to induce it to deform, for example, but not restricted to reverting to the original molded configuration or an approximation of that configuration.

### BACKGROUND OF THE INVENTION

In general this invention relates to implantable medical devices formed of shape-memory polymeric materials. More particularly, this invention is directed to catheters, stents, and shunts prepared from shape-memory polymeric materials and which can be deformed either prior to and/or during surgical procedures.

Cardiovascular diseases affect more than 2.5 million people each year. The number of affected individuals is expected to increase as the average age of the population increases. While the treatment regimes vary widely depending upon the nature and extent of the disease, common treatments include removing and repairing vascular occlusions, aneurysms, and ruptured or traumatized vessels. Currently, doctors often implant stents, catheters, shunts, stimulator leads, and the like to diagnose and treat many of these diseases. These devices are threaded through the vascular system to the treatment site. This often requires miniaturized, flexible surgical instruments, tubes and/or wires, and correspondingly small implants.

For example, localized occlusive arterial lesions, which can occur with atherosclerosis, are often treated with percutaneous transluminal angioplasty (PTA). A cardiologist might thread a guide catheter through the patent's vasculature to the site of the lesion. Once at the affected site, a balloon located within the catheter is inflated to compress the plaque against the arterial wall. Additionally, a small cutting tool can also be introduced through the catheter to physically cut away and remove some or all of the plaque. In some cases, often only a short time after the procedure, the arteries occlude either from re-occurrence of the plaque buildup or collapse of the arterial wall. Consequently, a cardiologist may elect to introduce a stent through the catheter. The stent can be expanded in the artery to press against the arterial wall and ensure that the artery stays open and allows sufficient blood flow. The catheter and balloon are eventually removed leaving the stent in place. Many stents are made a wire coil. Some stents include a shape-memory alloy material such as nitinol. However nitinol alloys have a limited ability to deform and expand. Currently, technology is limited to about an 8% volume change for nitinol alloys, which limits its ability to be used in stents introduced through the increasingly smaller catheters and incisions required for minimally invasive surgical techniques.

Vascular defects such as aneurysms and ruptures can also be diagnosed and/or treated using implantable catheters or stents. The stents can be used to reinforce vascular walls and/or secure grafts. The grafts can be used, for example, to repair a ruptured vessel or to connect two or more vessels together. The grafts can be obtained as autogenic tissue, allogenic tissue, or a synthetic material. Allogenic tissue requires a second surgery to harvest the tissue. The second surgical site often provides more pain and discomfort to the patient than the original or primary surgical site. This can impede a patient's recovery and even cause the patient to be reluctant to undergo these procedures. Alternatively, autogenic tissue grafts can be used. However, since the autogenic grafts are obtained from other sources, tissue compatibility and availability can be problematic. Additionally, there is always a chance of rejection and/or disease when an allogenic tissue graft is used. When suitable tissue is not available, for example, because of disease or extensive trauma, a synthetic graft or stent can be used in place of tissue graft. Obviously, the synthetic stents must be biocompatible and sufficiently flexible to be threaded to the target site. Selected polymeric materials can be used for stents or grafts. There continues to be increasing interest and development in this area to provide optimum stents/grafts. It would be desirable to provide a synthetic graft that is flexible or at least formable during surgery and yet provides sufficient support to repair the damaged vascular. Additionally, it would be desirable that if, and when, the stent is no longer needed, the stent can be eliminated without requiring a surgical revisit.

Thus, in light of the above described problems, there is a continuing need for advancements in the relevant field, including improved methods for treating patents, improved compositions for stents/grafts, and new, implantable, synthetic medical devices for the treatment of vascular disease and defects. The present invention is such an advancement and provides a wide variety of benefits and advantages.

### SUMMARY OF THE INVENTION

The present invention relates to a polymeric stent, the manufacture and use thereof. Various aspects of the invention are novel, nonobvious, and provide various advantages. While the actual nature of the invention covered herein can only be determined with reference to the claims appended hereto, certain forms and features, which are characteristic of the preferred embodiments disclosed herein, are described briefly as follows.

In one aspect the present invention provides a vascular stent comprising an elongated sleeve formed of a shape-memory polymeric material. The stent has an inner surface and an outer surface. The stent sleeve is provided in a first configuration such that the outer surface defines a fold along its length. A resistive wire is embedded in the stent between the inner and outer surface. When a small electric current is passed through the wire, the wire heats up. The stent absorbs the thermal energy radiating from the heated wire, and consequently, the sleeve is deformable to a second configuration that is different from the first configuration.

In general, the synthetic implant or stent is for use in medical treatment, for example, in treatment and/or repair of damaged and diseased vascular vessels. The stent is formed of a shape-memory polymeric material. The stent can be pre-formed in an original, desired shape or configuration and subsequently heated and deformed to provide the stent in a configuration that is readily implanted surgically. After the stent has been surgically implanted into the desired location, the stent can be heated or stimulated. As a consequence of the particular characteristics of the shape-memory polymeric material, the stent tends to revert to the original configuration or close approximation to that configuration. After the stimulated stent has reached the desired configuration, the stimulation or heat is removed. The stent then retains the newly acquired configuration whether the same as or substantially similar to the original configuration. In this fashion, the stent can remain in the body in the acquired configuration to facilitate repair and treatment of a damaged or diseased body.

In other embodiments, the polymeric material is resorbable and/or degradable so that the material can be safely reabsorbed or otherwise metabolized within the body over a period of time. This eliminates a necessity for subsequent surgical procedures to remove a stent that is no longer needed.

In another aspect the present invention provides a stent comprising: a body formed of a shape memory polymeric material. The body is provided in a first configuration defining a substantially planar ribbon having a first surface and an opposite second surface. A resistive wire is positioned on or in the ribbon proximate to the first surface, wherein said body upon application of an electrical current through said wire is deformable to a second configuration different from the first configuration.

In still yet another aspect, the present invention provides a method of treating patents in need of treatment, for example, patents with cardiovascular disease or damaged or defective vascular vessels or systems. The method comprises implanting a vascular stent into a vessel. The stent comprises an elongated sleeve formed of a shape-memory polymeric material and has an inner surface and an outer surface. A flexible, resistive wire is positioned on or in the stent. Application of an electrical current through the wire causes the wire to heat up. The polymeric material absorbs most of the energy from the heated wire. The temperature of the polymeric material increases to a level above its deformation temperature. Consequently, the sleeve is deformable to a second configuration that is different from the first configuration.

### BRIEF DESCRIPTION OF THE DRAWINGS

FIG. 1 is a perspective view of a tubular stent partially broken away in accordance to one embodiment of the present invention.

FIG. 2 is a perspective view of one embodiment of a deformed stent derived from the stent illustrated in FIG. 1 in accordance to the present invention.

FIG. 3 is an elevated first end view of the stent illustrated in FIG. 2.

FIG. 4 is perspective view, partially broken away, of a collapsed stent in accordance to another embodiment of the present invention.

FIG. 5 is an elevated first end view of a stent having multiple folds in accordance with another embodiment of the present invention.

FIG. 6 is a perspective view of a deformed stent having a rounded end in accordance to another embodiment of the present invention.

FIG. 7 is a side view in partial cross-sectional of a deformed stent positioned in a section of an artery in accordance with the present invention.

FIG. 8 is a side view in partial cross section of an expanded stent derived from the stent illustrated in FIG. 7 and positioned in a section of an artery in accordance with present invention.

FIG. 9 is a side view in partial cross section of a deformed stent inserted into an artery through a guide catheter in accordance with another embodiment of the present invention.

FIG. 10 is a side view in partial cross section of an expanded stent derived from the deformed stent illustrated in FIG. 9 in accordance with the present invention.

FIG. 11 is a side view in partial cross-section of a deformed stent positioned proximate to a vascular defect in accordance with the present invention.

FIG. 12 is a side view in partial cross section of an expanded stent derived from the stent illustrated in FIG. 11 in accordance with the present invention.

FIG. 13 is a perspective view of a mesh stent provided in a planar configuration in accordance with of yet another embodiment the present invention.

FIG. 14 is a perspective view of a mesh stent derived from the sent illustrated FIG. 13, but deformed into a helical configuration.

FIG. 15 is a perspective view of a deformed ribbon stent in accordance of yet another embodiment of the present invention.

FIG. 16 is a perspective view of an deformed stent derived from the sent illustrated in FIG. 15.

### DETAILED DESCRIPTION OF THE INVENTION

For the purposes of promoting an understanding of the principles of the invention, reference will now be made to the embodiments illustrated herein and specific language will be used to describe the same. It will nevertheless be understood that no limitation of the scope of the invention is thereby intended. Any alterations and further modifications in the described implants, stents, devices and/or processes, and any further applications of the principles of the invention as described herein, are contemplated as would normally occur to one skilled in the art to which the invention relates.

In general the present invention provides an axially expanding, polymeric stent. The polymeric stent is preferably made of a shape-memory polymeric (SMP) material that is originally molded into a desired shape or configuration. The molded stent can be deformed upon the application of a selected stimuli, such as, heat, radiation, inducing a pH change, or a combination thereof. Typically heat is used to stimulate an SMP material. A shape-memory polymeric material becomes plastic above its glass transition temperature, T_{g}. This allows the molded stent to be readily deformed to another configuration simply by heating the shape-memory polymeric material above its T_{g}. Cooling the material below T_{g} effectively freezes the stent in the deformed configuration. However, if the material is reheated above T_{g}, the material again becomes plastic, and the stent tends to automatically revert to its original, molded configuration—in the absence of any applied or external shape constraining pressure(s). A stent formed of an SMP material can be molded or deformed to conform to a desired anatomical configuration for treatment. The shape-memory polymeric material can be selected and/or modified to exhibit a T_{g} at a pre-selected temperature level or range. Additionally, the shape-memory polymeric material can be selected to provide a stent that can retain sufficient rigidity and/or resiliency to support surrounding tissue as necessary. Further, the shape-memory polymeric material can have varying degrees of plasticity above T_{g}.

As used herein, the term stent is intended to include any tubular implantable medical device suitable for the treatment of humans and other animals.

It should be understood that the implantable devices and methods described herein are not restricted to the diagnosis and treatment of cardiovascular diseases. In general, the use of the stent as described herein for the diagnosis and treatment of any disease is contemplated to be within the scope of the present invention.

In one embodiment, the polymeric stent is originally molded in a desired configuration or shape suitable to effect the desired treatment. For example, the stent can be molded into a cylindrical sleeve sized to either replace a portion of a vein or artery or be implanted into a vein or artery. In other embodiments, the stent can be molded to serve as a connector or graft for one, two, or more vascular vessels, lymphatic vessels, or other membranous canals, such as the urethra. As used in this application, the term vascular includes within its scope vessels or ducts that convey fluids, for example body fluids such as blood, lymph, or urine in humans and other animals.

The originally molded stent is deformed prior to implantation. Usually a deformed configuration is selected to facilitate implantation into a patent. Preferably the polymeric material is deformed into a shape and/or configuration that permits facile insertion using minimally invasive surgical techniques. Typically this involves implantation through small incisions and/or threading the formed stent through arteries and/or veins. Therefore, the deformed stent usually has a smaller cross-sectional area or profile than the original molded configuration. The deformed stent can be implanted using standard catheter techniques, for example, through a percutaneously placed sheath inducer, which is well known in the art. The deformed stent can be implanted into a vein or artery through a small guide catheter or sheath during surgical treatment, for example, during a (TBA) procedure. The deformed stent can be guided or threaded to the desired treatment site using fluoroscopy to ensure the correct placement and/or functioning of the stent.

Either prior to, during, or after implantation, the deformed stent is subjected to conditions that will induce the deformed stent to return to its original molded configuration. Typically this entails heating the shape-memory polymeric material to a temperature level above its T_{g} value. Heating above the T_{g} value for the selected shape-memory polymeric material can stimulate the stent (or more specifically the polymeric material used to form the stent).

Heating the shape-memory polymeric material can be accomplished through a wide variety of techniques and procedures. Such techniques include, but are not restricted to, heating with a warm water or saline solution (preferably sterile), electrical heat, light, and/or radiation. The warm water can be injected into or circulated through the catheter to heat the material. Alternatively, an electrically heated wire, a miniature light, a laser, or other radiation source can be threaded through the catheter to stimulate the shape-memory polymeric material. In a selected embodiment, the stent includes a small, flexible, resistive wire attached to or embedded within the shape-memory polymeric material.

Stimulating the shape-memory polymeric material induces the stent to return to its original, molded configuration or a close approximation thereof. Alternatively, the stent can be deformed to a second configuration induced by the surrounding tissue or selected by the surgeon for the desired effect either for treatment or diagnosis. Typically the newly acquired configuration is an expanded configuration. For example, the expanded configuration can be used as a graft and/or to maintain and support a vessel following dilation and/or removal of lesions using angioplasty procedures. Once the implanted stent has either been deformed or allowed to revert to its original configuration, the polymeric material is allowed to cool below its T_{g} temperature value to effectively freeze the stent in the newly acquired configuration.

In a preferred embodiment, a balloon positioned inside the deformed stent can be expanded to force the stent into a desired configuration and to balance any contracting forces exerted by the neighboring tissue. This can be accomplished, for example, by using the same balloon techniques used in transluminal balloon angioplasty procedures. Alternatively, the stent can be expanded under air or water pressure introduced through the catheter, or otherwise bent to a desired second configuration using a resilient wire probe.

Once the stent has been implanted and deformed to a desired configuration, it is separated from the catheter. This can be accomplished using techniques commonly used to release embolic coils into the vascular system. Alternatively, the shape-memory properties of the material can be used advantageously to provide a release mechanism. For example, initially the stent can be deformed to grip or otherwise engage a guide catheter or wire. After heating, the stent deforms, preferably by expanding, and as a result of the deformation releases or disengages the catheter or wire. In other embodiments, the stent can be detached from the catheter or guide wire either prior to or after it has been initially deformed in the body.

The stent remains in the vascular vessel maintaining the opening while healing and/or regrowth occur. In the preferred embodiments, the shape-memory polymeric material is a biodegradable material that can degrade within the body over a period of time. This biodegradable shape-memory polymeric material does not require a second surgical procedure to remove the stent or graft when it is no longer needed.

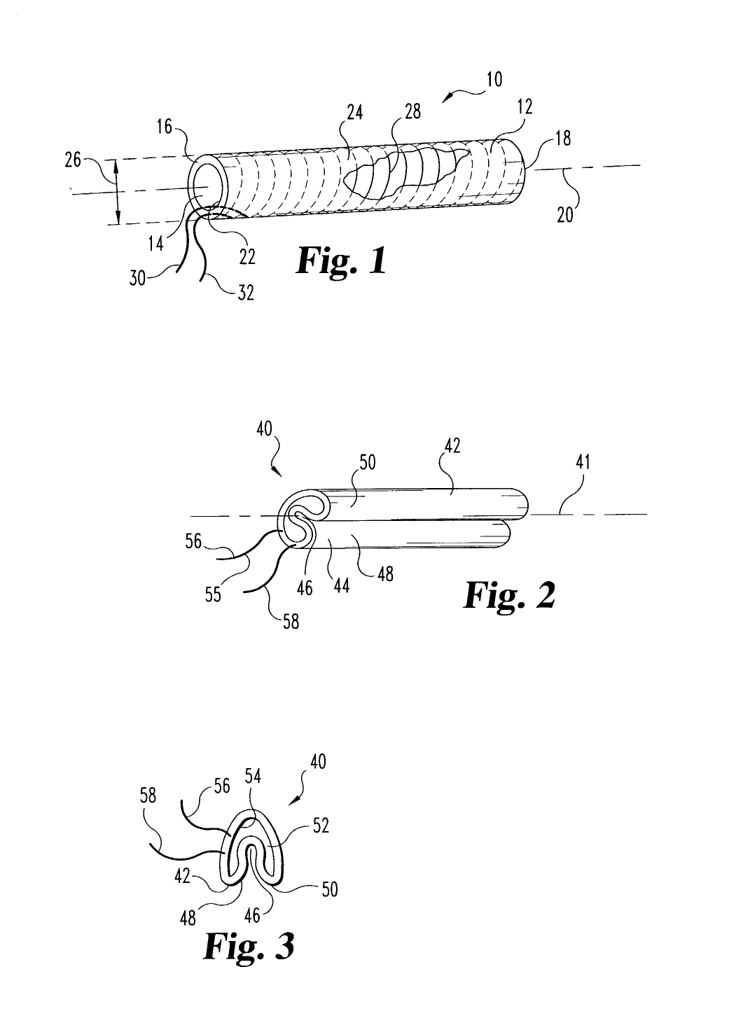

FIG. 1 illustrates one embodiment of a stent 10 according to the present invention. Stent 10 is provided in a configuration of a sleeve 12 having a passageway 14 therethrough. Sleeve 12 includes a first end 16 and an opposite second end 18 providing openings into passageway 14. Sleeve 12 is provided as a substantially elongated cylinder defining a longitudinal axis 20. Passageway 14 extends through sleeve 12 along the longitudinal direction. Sleeve 12 includes an inner surface 22 and an exterior surface 24. In the illustrated embodiment, sleeve 12 is imperferate. In alternative embodiments, sleeve 12 can include a plurality of openings or pores formed through inner surface 22 and exterior surface 24. Exterior surface 24 defines an outer diameter represented by reference line 26. The outer diameter of stent 10 can be selected to be useful for the desired application. For example, the outer diameter of stent 10 can be selected to range between the desired interior diameter of a vein or artery such as commonly found in the body. In other embodiments, the diameter 26 can be selected to be at least as great as 1 mm, more preferably greater than 2 mm. In other embodiments, the diameter of stent 10 can be provided to be less than 15 mm, more preferably less than 12 mm.

The length of stent 10, measured along longitudinal axis 20, can vary depending upon the desired application. In preferred embodiments, the length is selected to be at least as great as 0.5 cm, more preferably at least as great as 2 cm. In alternative embodiments, stent 10 can provided as a long tubular structure. During surgery, the surgeon can select the desired length of the tubular structure for the desired application. Since stent 10 is formed of a polymeric material, this polymeric material is readily cut and/or shaped by the surgeon during surgery. Therefore, during surgery, the surgeon can measure a desired length of stent 10 and cut the desired length from the rest of the tubular stent material.

Additionally, stent 10 can be deformed, repeatedly if necessary, by the surgeon prior to implantation. For example, the stent can be submerged in a warm water bath maintained at a sufficiently high temperature level to heat the shape-memory polymeric material above its T_{g} value. While the temperature level of the shape-memory polymeric material is sufficiently high, the surgeon can mold or deform the stent into a desired configuration. Cooling the deformed stent below the temperature level equal to T_{g} effectively freezes the stent into the deformed configuration. The deformed stent can then be implanted into the patent. In a preferred application the deformed stent allows the surgeon to use minimally invasive surgical techniques to implant the stent.

In selected embodiments, stent 10 includes a resistive heating element, for example, wire 28 substantially encased within the polymeric material. Wire 28 is visible through the partially broken away section of sleeve 12. Wire 28 is suitably flexible to readily bend as stent 10 is deformed. Wire 28 is resistive and becomes warm when an electrical current flows therethrough. Wire 28 includes a first lead 30 and a second lead 32. Leads 30 and 32 can be connected to an electrical circuit before or during surgery.

Wire 28 extends into stent 10 and is selectively positioned and sized to extend substantially along the entire length of stent 10. Wire 28 can be substantially enclosed with stent 10 positioned between inner surface 22 and outer surface 24. Alternatively, wire 28 can be positioned on or substantially proximal to inner surface 22. In still other alternatives, wire 28 can be positioned near outer surface 24.

During surgery or after implantation, stent 10 is subjected to one or more stimuli to allow the polymeric material to deform. Typically this involves heating stent 10 to raise the temperature of the polymeric material above the temperature level equal to its glass transition temperature, T_{g}. Wire 28 provides a means for heating stent 10 above the deformation temperature of the shape-memory polymeric material. As current passes through wire 28, it heats up. The SMP absorbs the thermal energy from wire 28. If the thermal energy is sufficient high, then the polymeric material becomes plastic. When in the plastic state, stent 10 either reverts to its original configuration or readily deforms to another configuration with a minimum applied force.

Embedding wire 28 in the SMP or near surface 22 provides distinct advantages. As wire 28 heats up, the thermal energy is absorbed by the SMP. The SMP, in effect, insulates the neighboring tissue from the warm or hot wire 28. This protects the neighboring tissue from becoming too hot and can significantly reduce tissue damage.

Either during manufacture, immediately prior to surgery, or during surgery, the shape-memory polymeric material can be heated and thereafter deformed into a shape to facilitate implantation and/or treatment of a patient.

FIG. 2 illustrates one embodiment of the deformed stent 40. Deformed stent 40 is derived from stent 10. Subjecting the shape-memory polymeric material to stimuli such as heat and thereafter applying pressure to force the stent into a first deformed configuration forms deformed stent 40. In the illustrated embodiment, deformed stent 40 is provided as a substantially elongated sleeve 42 having a “U-shaped” cross section. When compared to stent 10, sleeve 42 exhibits a reduced cross-sectional area or profile measured or viewed transverse to a longitudinal axis 41. Sleeve 42 has an exterior surface 44. A fold 46 in exterior surface 44 extends along the direction of longitudinal axis 41. Fold 46 is defined by a first portion 48 of the exterior surface 44 positioned proximate to a second portion 50 of exterior surface 44. First portion 48 may, but need not, contact second portion 50. At ambient temperature, deformed sleeve 42 maintains the “U-shaped” configuration without the necessity of any added securing means, such as a clamp, suture or glue line, or bead. In one consideration, the “U-shaped” configuration allows fluid flow around the exterior and through the channel defined by fold 46 along the exterior of sleeve 42. This can be used to ensure that blood flow continues through the vein or artery during implantation of deformed stent 40.

Stent 40 also includes a resistive wire 55. Wire 55 can be embedded in the SMP material. Alternatively, wire 55 can be positioned internally in stent 40 proximate to an inner surface. Wire 55 is a continuous wire beginning at lead 56, entering a proximate end of stent 40, and extending substantially to the distal end before returning and exiting from the proximate end as lead 58. Wire 55 can spirally wind about stent 40. Alternative wire 55 can follow a boustrophedonic pattern along the length of stent 40.

Referring additionally to FIG. 3, an elevated, first end or proximal view of deformed stent 40 is illustrated. As can be readily seen from this illustration, deformed stent 40 includes a passageway 52 extending along its length. Deformed stent 40 is not deformed or compressed to cause internal surface 54 to completely collapse onto itself. Passageway 52 allows various objects or fluids to extend into and flow through deformed stent. This provides particular advantages during selected surgical procedures. For example, passageway 52 allows the insertion of a guide wire or an expandable balloon into deformed stent 40. The surgeon can use fluoroscopic techniques to thread a guide wire with an attached stent through the vasculature to the treatment site. Additionally, wire 55 is radio-opaque and can be readily visualized using standard techniques during and after surgery. A balloon positioned inside deformed stent 40 can be expanded after the shape-memory polymeric material has been heated about its T_{g}. Passing a small current of electricity through wire 55 causes it to heat up and warms the SMP above the deformation temperature. Expansion of the indwelling balloon can be used to expand the stent and counteract contracting forces exerted by the surrounding tissue.

In alternative embodiments, deformed stent 40 does not include a passageway 52 therethrough. This stent embodiment finds advantageous use where flow through or insertion through the stent is not required or desired. In one consideration, a stent without a thru passageway can be used to facilitate implantation. This technique is similar to those catheterization techniques using a flow-directed, balloon-tipped catheter inserted into a pulmonary artery. The deformed stent can be used in addition to or as a replacement for the small balloon. In this embodiment, the deformed catheter may include a closed or partially closed passageway. A closed passageway can be formed, for example, by pinching or otherwise closing off one end of deformed stent 40.

In other embodiments, a deformed stent can be provided with more than one fold extending along its longitudinal axis. FIG. 4 illustrates one example of a deformed stent 60 having two folds along its longitudinal axis. Deformed stent 60 includes a deformed sleeve 62 having a first fold 64 and an opposite second fold 66 extending along its longitudinal direction. Stent 60 also includes a wire 68 as a resistive heating element.

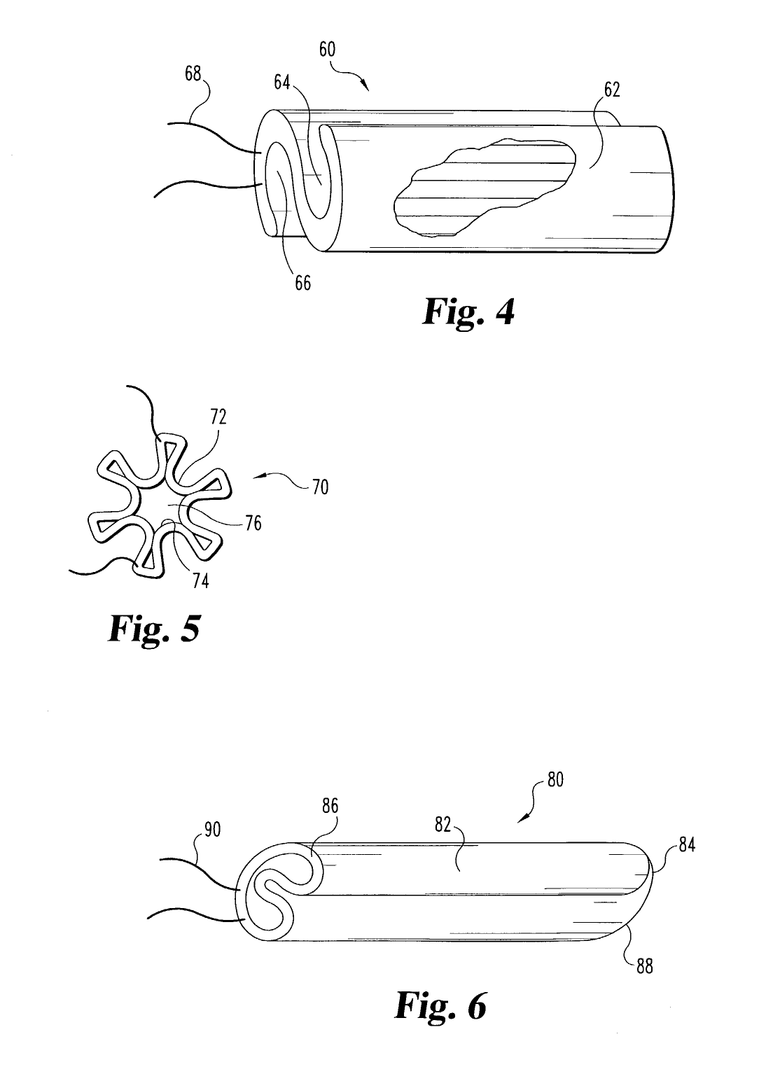

FIG. 5 illustrates an elevated first end view of a deformed stent 70 having a plurality of folds 72 extending along its longitudinal axis. Deformed stent 70 can be envisioned to resemble a circular pleated sheet or an accordion. Stent 70 includes an interior surface 74 defining a passageway 76. Passageway 76 extends through deformed stent 70. As has been described in earlier embodiments, deformed stent 70 need not include a passageway extending completely through the sleeve. Rather in other embodiments, interior surface 74 can collapse back on itself to block any flow of fluids through the interior of deformed stent 70. Alternatively, deformed stent 70 can include more than one passageway extending therethrough. Stents with more than one passageway can be used in combination with a duel lumen catheter, where each of the lumens are directed to or connected to the separate passageways.

FIG. 6 illustrates yet another embodiment of a deformed stent 80 for use in the present invention. Deformed stent 80 includes elongated sleeve 82 having a first end 84 and opposite second end 86. First end 84 is deformed to have a conical or convexly curved surface 88. Surface 88 provides a streamlined profile to stent 80. The streamlined profile can enhance the ease of insertion of stent 80 into the vascular system and/or a catheter sheath. Other embodiments for surface 88 can also be envisioned for the present invention including a wedge, a frustoconical surface, rounded or blunt surface.

Opposite end 86 is provided substantially as has been described for deformed stent 40, although it will be understood by those skilled in the art that opposite end 86 can also be provided substantially as has been described for first end 84. Stent 80 can have one, two, or more folds extending along its longitudinal direction. Additionally, stent 80 includes a resistive heating element 90 provided as a small, flexible wire substantially encased within the SMP material.

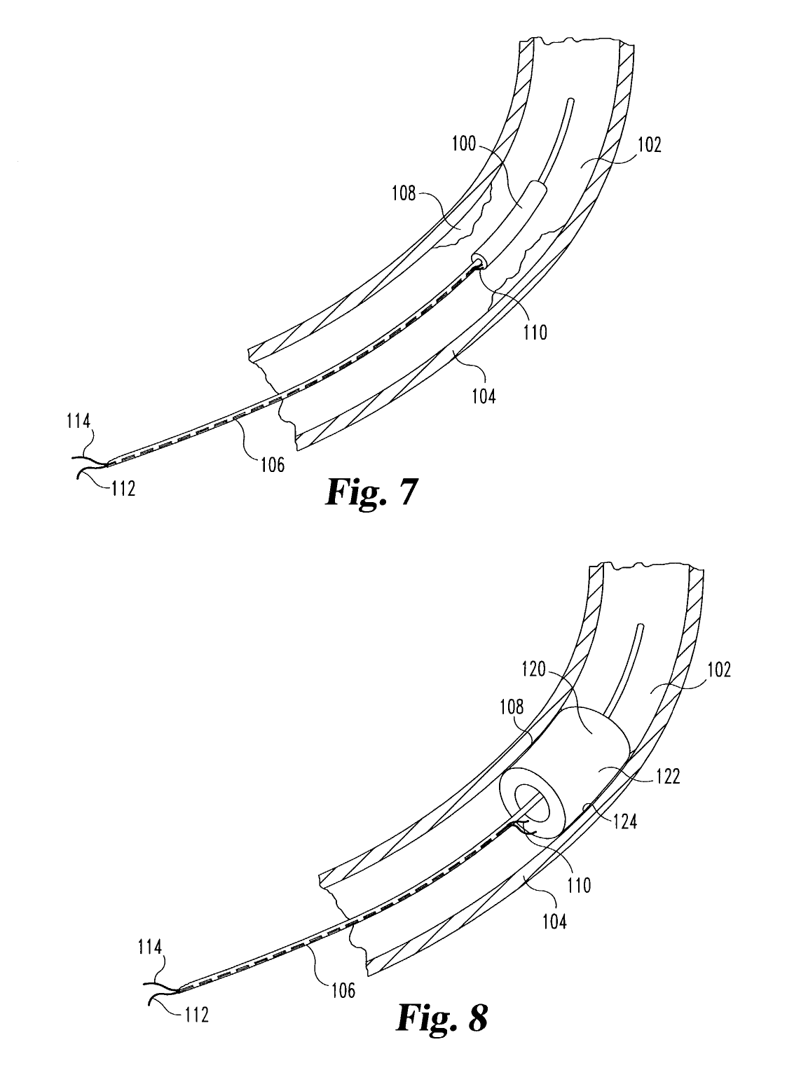

FIG. 7 is an illustration of a deformed stent 100 positioned within an vascular vessel 102 bounded by tissue 104. Deformed stent 100 is removably attached to guide wire 106, which has been threaded through the vascular system from a remote incision site. In the illustrated embodiment, stent 100 has been positioned proximate to a lesion 108 formed on vessel 102. Stent 100 includes a flexible, resistive wire 110 having leads connected to an electrical source. Preferably, the electrical source includes a pair of wires 112 and 114 extending along guide wire 106. In preferred embodiments, wire 110 is releasable from wires 112 and 114. In other embodiments, wire 110 remains attached to wires 112 and 114. Wires 112 and 114 extend through or along guide wire 106 to a power supply typically located near the patient during the operation.

In use, after stent 100 has been positioned in the vasculature as desired, a small electric current is passed through wire 110 via wires 112 and 114. Since wire 110 is resistive, it heats up, and the SMP material proximal to wire 110 absorbs the thermal energy. When the SMP has absorbed sufficient energy to reach a temperature level equal to or above the SMP's deformation temperature, stent 100 deforms to provide stent 120, illustrated in FIG. 8.

Deformed stent 120 is in a second configuration, which is derived from deformed stent 100. Consequently, the same reference numbers used in FIG. 7 will be used to identify the same structures or features. Deformed stent 120 includes an outer surface 122, which bears against the inner surface 124 of tissue 104. It should be understood that in a selected embodiment, the configuration of deformed stent 120 is equal to or approximates the original molded configuration of an original molded stent, for example, stent 10 described above. The constrains imposed by the surrounding tissue—either tissue 104 or adjacent tissue external to tissue 104 (not shown)—can influence the configuration of stent 120. For example, the interior diameter of vessel 102 can restrict the outer diameter of stent 120. Since stent 120 bears against and exerts pressure against the vascular tissue 104, stent 120 is secured in place without the necessity of sutures or other fastening means. In an alternative embodiment, stent 120 includes a plurality of openings through surface 124 through which tissue can be forced or grow into. Further, stent 120 can be used to maintain an opening through vessel 102 by ensuring that the lesion(s) remain compressed against tissue 104 or by supporting any resected tissue, for example, previously provided by a TBA technique.

After deformed stent 120 has been placed in the desired location, guide wire 106 can be removed using known techniques. In one embodiment, application of the selected stimuli to deformed stent 100 both deforms stent 100 to the second configuration 120 and at the same time releases guide wire 106 and wire 110. Guide wire 106 can then be withdrawn from the vascular system, as desired, leaving deformed stent 120 at the treatment site. In other embodiments, wire 110 remains connected to lead wires 112 and 114. When guide wire 106 is withdrawn, lead wires 112 and 114 and wire 110 are also withdrawn. This leaves expanded stent 120 positioned inside the vasculature. This is particularly advantageous when wire 110 is positioned internal of stent 100/120 so that removal of the combined wires 112, 114, and 110 does not displace expanded stent 120.

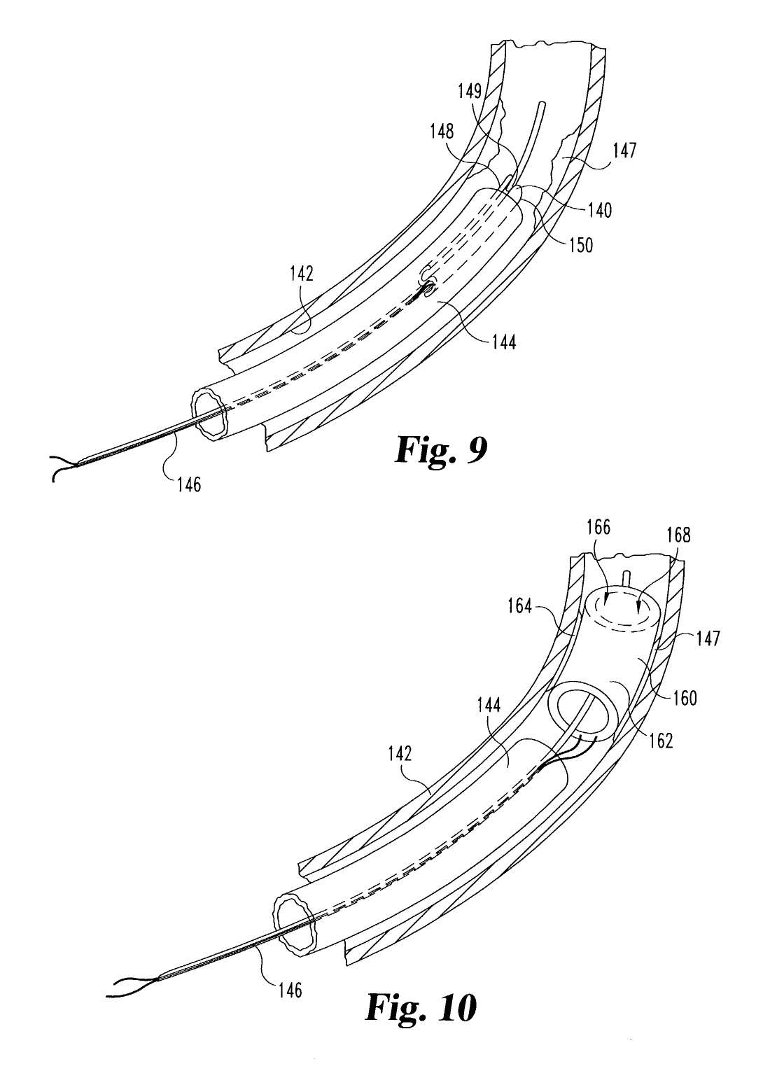

FIG. 9 illustrates yet another embodiment of the current invention. Deformed stent 140 is introduced to the vascular vessel 142 via a dilation catheter system 144 including a guide catheter or wire 146. Deformed stent 140 is derived from stent 80 and includes a convexly curved surface 148 on a first end 149. In the illustrated embodiment, stent 140 does not provide a thru passageway. Once dilation catheter 144 has reached an area proximate to the treatment site 147, further insertion of dilation catheter 144 is arrested. Guide catheter 146 and attached stent are positioned inside dilation catheter system 144 either prior to its insertion in the vascular system or after it has been placed proximal the treatment site 147. Extension of guide catheter 146 forces deformed stent 140 out from inside dilation catheter 144 such that the external surface 150 of stent 140 is positioned opposite desired treatment site 147. Once the deformed stent 140 has been placed in the desired position, deformed catheter 140 is stimulated to induce deformation, for example axial expansion, to provide the expanded catheter.

FIG. 10 illustrates expanded stent 160 derived from stent 140 from FIG. 9. Consequently, the same reference numbers used in FIG. 9 will be used to identify the same structures or features. Stent 160 expands and substantially fills the interior of vessel 142. External surface 162 bears against the interior wall 164 of vessel 142. Catheter 160 includes an interior passageway 166 extending therethrough. It will be noted that the convexly curved surface 148 of stent 140 has now been deformed to provide an opening 168 to passageway 166 and allows fluid flow through stent 160. Once the surgeon has determined that stent 160 is sufficiently secured at the treatment site 147, guide catheter 146 and dilation catheter 144 can be removed from the vessel 142.

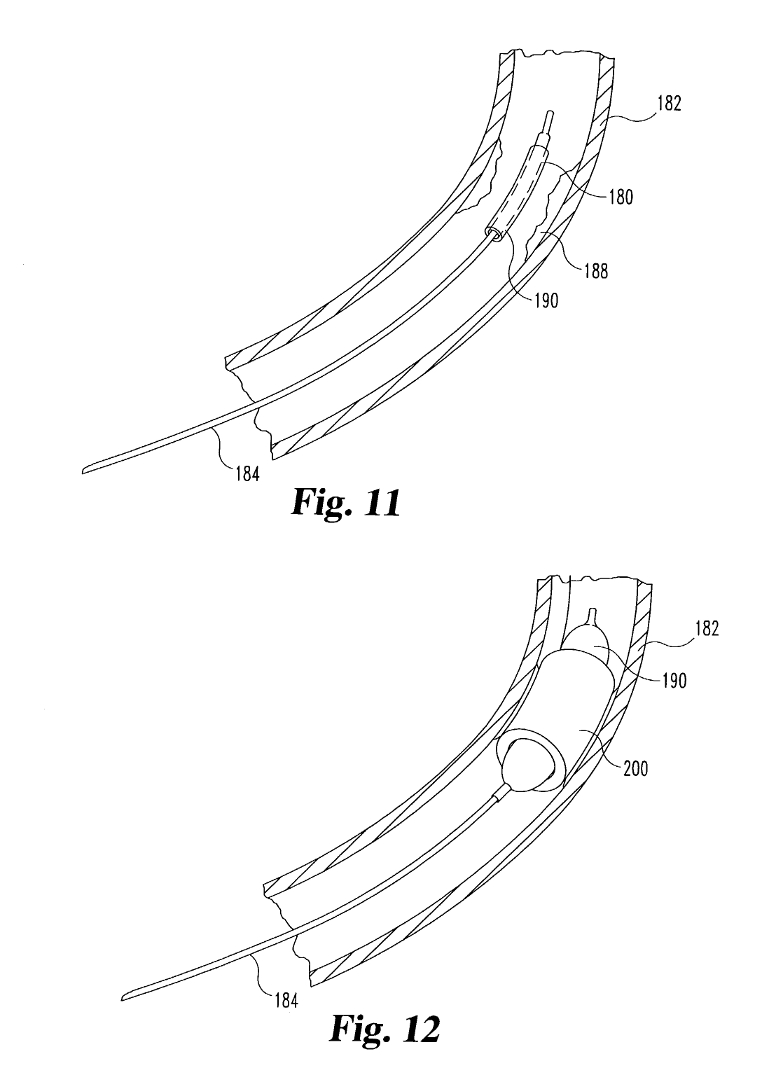

FIG. 11 illustrates yet another embodiment of an expandable stent 180 according to the present invention. Expandable stent 180 can be inserted into a vascular vessel 182 using a guide wire or catheter 184 to position stent 180 opposite a desired site 188 in vascular vessel 182. A deflated balloon 190 is positioned inside stent 180.

FIG. 12 illustrates expanded stent 200 derived from stent 180. Consequently, the same reference numbers used in FIG. 11 will be used to identify the same structures or features. Stent 200 has been subjected to or absorbed thermal energy, for example by filling balloon 190 with a warm saline solution maintained at a temperature level greater than T_{g} for the selected shape-memory polymeric material forming stent 200. The warm saline solution can be introduced into balloon 190 through catheter 184, which can include one or more lumens (not shown). Two lumens are preferred to allow circulation of the warm saline solution through the system. Once stent 200 has been sufficiently heated to a temperature level greater than T_{g}, the shape-memory polymeric material becomes plastic and can be readily deformed. Alternatively, or in addition, stent 200 can include one or more resistive heating elements such as a small, flexible wire. An electrical current can be passed through the wire as discussed above. The SMP will absorb the thermal energy from the wire. In one aspect, stent 200 self deforms or reverts to is original molded configuration, for example, a configuration similar to stent 10. In another aspect, balloon 190 can be expanded using hydrostatic pressure to expand balloon 190, and consequently, stent 200 to a desired deformed configuration. The desired configuration can approximate the original molded configuration or it can vary from the original molded configuration. For example, it is possible to expand stent 200 to have an outer diameter greater than the outer diameter of the original molded configuration. Once the desired configuration has been acquired, the temperature level of the saline solution can be reduced below T_{g}. This effectively freezes stent 200 in the desired configuration. The hydrostatic pressure in balloon 190 can be reduced allowing balloon 190 to deflate or collapse. The deflated balloon readily releases stent 200 to be retained within the vascular vessel 182. The surgeon subsequently removes deflated balloon 190, as desired, to complete the procedure.

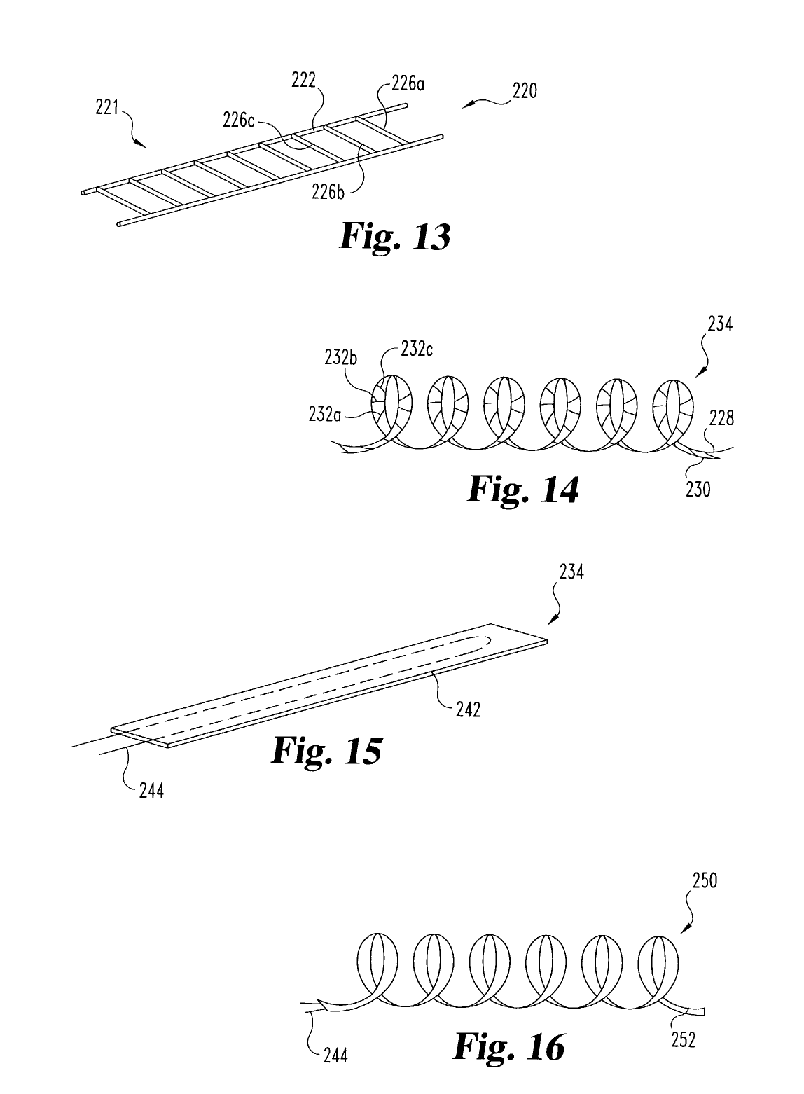

FIG. 13 illustrates yet another embodiment of a deformed stent 220 for use in the present invention. Stent 220 is formed as a substantially flat lattice 221 illustrated as having two substantially parallel legs 220 and 224. Alternatively, stent 220 resembles a substantially planar, mesh ribbon. A plurality of cross members or rungs 226a, 226b, 226c, . . . extend between leg 222 and leg 224. It will be understood by those skilled in the art that a lattice 221 can include other configurations having more than two substantially parallel legs and any number of cross members 226. Furthermore, while cross members 226 are illustrated as substantially parallel to each other and substantially perpendicular to one or the other leg 222 or 224, alternative configurations are envisioned and are intended to be included in the present invention. Lattice 221 is formed substantially of a shape-memory polymeric material. As with the other embodiments of the present invention, stent 220 can include a heating element. The heating element can either be imbedded within the SMP material or positioned adjacent to one or more members of the stent.

The shape-memory polymeric material can be provided in the form of a lattice using extrusion techniques that are well known in the art. The shape-memory polymeric material is provided to have a deformation temperature suitable for use as a stent in a vascular system. In this regard, the deformation temperature of the SMP should be sufficiently low such that heating the material to its deformation temperature will not injure or destroy adjacent tissue. When stent 220 is provided as a substantially planar structure it can be readily inserted into a small tubular guide wire that has been threaded through the patient's vascular system to a desired lesion for treatment or repair.

Prior to insertion into the patient, either at the place of manufacture or immediately before surgery, stent 220 is formed. Stent 220 can be formed by heating the implant as originally molded above the deformation temperature of the SMP and forcing the original molded implant into a substantially planar configuration. It should be further understood that, if desired, stent 220 can be folded along its longitudinal axis. This in effect would double the plurality of rungs 226 upon themselves and position leg 222 on top of leg 224. This would further reduce the cross-sectional area of stent 220 to facilitate insertion into the vascular system.

FIG. 14 illustrates a stent 232 as originally molded that can be used to provide stent 220. Stent 232 is illustrated as a double helix 234 having a pair of substantially parallel legs 228 and 230. A plurality of cross members 232a, 232b, 232c, . . . extend from leg 228 to leg 230. In selected embodiments, stent 232 helical angle of between about 20° and about 50 more preferable between about 30° and about 50°.

FIG. 16 illustrates still yet another embodiment of a stent 240 for use in the present invention. Stent 240 is illustrated as a substantially planar ribbon 242. In the illustrated embodiment, planar ribbon 242 is an imperforate ribbon. Additionally, ribbon 242 can include a resistive wire 244 positioned on or extending therethrough. In other embodiments, ribbon 242 need not include the resistive wire. Stent 240 is provided in a first, deformed configuration that exhibits a substantially reduced cross-sectional area or profile to be readily inserted through a catheter guide wire into the vasculature.

FIG. 17 illustrates a deformed stent 250 derived from stent 240. Stent 250 is provided by heating stent 240 above its deformation temperature. Upon heating above the deformation temperature, the SMP automatically reverts to its original, molded configuration, which is illustrated as stent 250. Stent 250 is illustrated as a helical coil that can be used as a stent to open substantially closed or blocked arteries, repair damaged arteries, and/or replace sections of arteries. In preferred embodiments, the helical angle can be provided to have an angle between about 30° and about 50°, more preferably between about 40° and about 45°.

In preferred embodiments, the stents illustrated in accordance with this invention, including catheters 10, 40, 80, 100, 120, 140, 160, 220, 229, 240, and 250, are formed from a shape-memory polymeric material. The shape-memory polymeric material can be or include a bio-absorbable material such as a polylactic acid (PLA) or a combination of polyglycolic acid (PGA) and PLA. Examples of polymeric materials suitable for the present invention include both biodegradable and non-biodegradable polymers. In preferred embodiments, the shape-memory polymeric material is formed from oligomers, homopolymers, copolymers, and polymer blends that include polymerized monomers derived from l, d, or d/l lactide (lactic acid); glycolide (glycolic acid); ethers; olefins, such as ethylene, propylene, butene-1, pentene-1, hexene-1, 4-methylpentene-1, styrene, norbornene and the like; butadiene; polyfunctional monomers such as acrylate, methacrylate, methyl methacrylate; esters, for example, caprolactone, hydroxy buteric acid, hydroxy valeric acid; and mixtures of these monomeric repeating units.

Use of the term copolymers is intended to include within the scope of the invention polymers formed of two or more unique monomeric repeating units. Such copolymers can include random copolymers, graft copolymers, block copolymers, radial block, diblock, triblock copolymers, alternating copolymers, and periodic copolymers. Use of the term polymer blend is intended to include polymer alloys, semi-interpenetrating polymer networks (SIPN), and interpenetrating polymer networks (IPN).

Preferred shape-memory molded stents of this invention are fabricated to include homopolymers, copolymers, polymer blends, and oligomers of d, l, d/l, polylactide; polyglycolide, poly(lactide-co-glycolide), poly(β-hydroxy butyrate); polyβ-hydroxy butyrate-co-hydroxyvalerate), (poly(trimethylene carbonate) polyurethane, poly(ethylene-co-vinyl acetate) (EVA), poly(ethylene-co-propylene) (EPR), poly(ethylene-co-propylene-co-diene) ter-polymer (EPDM), poly(ε-caprolactone), poly imino carbonates polyanhydrides, copolymers of ethylene and propylene and/or other α-olefins: or copolymers of these α-olefins. Among these, various types of polyethylene, such as low-density polyethylene, linear low-density polyethylene, medium-density polyethylene and high-density polyethylene, and polypropylene are preferable.

Preferred polymers include biodegradable homopolymers of lactide or glycolide or copolymers thereof. Exemplary polymers are described in U.S. Pat. No. 4,950,258, the entire disclosure of which is incorporated by reference herein. When copolymers of lactide and glycolide are used to form the molded products, the copolymers preferably consist essentially of a composition of 90-10 mol % lactide and 10-90 mol. % glycolide, and most preferably consist essentially of 80-20 mol. % lactide and 20-80 mol. % of glycolide. Within these specified ranges, the copolymers exhibit desirable deformation characteristics. For example, the copolymers are more pliable and readily deformable at lower temperatures when their mole ratio of lactide and glycolide approximates to 1:1. Generally, the less crystalline phases in the shape-memory polymeric material, the lower the deformation temperature.

The polymer composition of the present invention may further contain thermoplastic resins and/or thermoplastic elastomers to improve its stiffness, moldability, and formability. In addition, the shape-memory molded stent may additionally include additives such as coloring agents, stabilizers, fillers, and the like, in an amount such as will not alter the desired shape-memory effect, biocompatibility, and/or biodegradability properties of the molded stents.

The polymer is characterized in that it will attempt to assume its memory condition by activation of a polymer transition. Activation can occur by adsorption of heat by the polymer or a change in pH in the liquid in contact with the polymer. Incorporation of a material such as methacrylic acid or acrylic acid into the polymer results in a polymer having a transition that is sensitive to pH. The polymer transition may be a thermally-activated transition, whereupon adsorption of heat the polymer undergoes a glass transition or a crystalline melting point.

When polymers such as biodegradable polymers are provided with less crystallinity, they degrade at a much faster rate than polymers that have greater degrees of crystallinity. Polymers with a lesser degree of crystallinity can be prepared by providing copolymers of lactic acid and galactic acid. Increasing the amount of galactic acid in the polymer decreases its crystallinity and therefore increases its rate of degradation.

As mentioned above, the molded stent can be deformed when heated above its glass transition temperature, T_{g}. When heated above its T_{g}, the polymeric material exhibits an elasticity or super elasticity that allows it to be molded into a variety of shapes. For example, for the present invention, the molded stent can be heated to a temperature between about 35° and about 100° C. Preferably the shape-memory polymeric material is selected to have a T_{g} of at least 35° C., more preferably at least 45° C. The higher range T_{g} temperature level for the shape-memory polymeric material can be less than the temperature that causes tissue damage, more preferably less than about 100° C., more preferably less than about 80° C., still more preferably less than about 70° C. Application of a compressive force to deform the stent into a deformed configuration having a reduced cross-sectional profile can then be applied. The deformed stent can then be cooled below the T_{g}, which effectively freezes the deformed implant into its deformed configuration. The deformed stent can be used immediately or stored and/or shipped for use at a later time. Obviously, prior to use, the deformed stent should be sterilized, preferably using chemical or radiation sterilization techniques.

The various embodiments of stents for use in the present invention can be prepared by extruding the desired polymeric material. For example, a desired polymeric precursor can be extruded on to a mandrel. The mandrel can include thereon a resistive wire, which can be embedded in or adhered to the resulting structure. In preferred embodiments, the polymeric material is extruded on to the selected mandrel and then heated with or without added pressure to cure the material and form the stent in the original, molded configuration.

The foregoing illustrates and describes various embodiments for stents and implants for use in the present invention. One or more of the features or structures described herein for any of the individual embodiments of the present invention can be combined with other features, structures and/or alternative embodiments of this invention, and as such ,are intended to be within the scope of the present invention.

The present invention contemplates modifications of the illustrated embodiments as would occur to those skilled in the art. It is also contemplated that the stents, implants, and processes embodied in the present invention can be altered, rearranged, substituted, deleted, duplicated, combined, or added to other processes as would occur to those skilled in the art without departing from the spirit of the present invention.

All publications, patents, and patent applications cited in this specification are herein incorporated by reference as if each individual publication, patent, or patent application was specifically and individually indicated to be incorporated by reference and set forth in its entirety herein. Further, any theory of operation, proof, or finding stated herein is meant to further enhance understanding of the present invention and is not intended to make the scope of the present invention dependent upon such theory, proof, or finding.

While the invention has been illustrated and described in detail in the drawings and foregoing description, the same is considered to be illustrative and not restrictive in character, it is understood that only the preferred embodiments have been shown and described and that all changes and modifications that come within the spirit of the invention are desired to be protected.

What is claimed is:

1. A vascular stent comprising:
an elongated sleeve formed of a shape-memory polymeric material and having an inner surface and an outer surface;
a resistive heating element positioned proximate the inner surface and the outer surface;
said sleeve provided in a first configuration wherein the outer surface is deformed to define a fold and, upon application of selected stimuli, said sleeve is deformable to a second configuration different from the first configuration.

2. The stent of claim 1 provided in an original configuration as a substantially cylindrical sleeve defining a hollow interior space and having a first end and an opposite second end, said first and second ends providing openings into the interior space.

3. The stent of claim 2 wherein the second configuration approximates a stent in the original configuration.

4. The stent of claim 1 wherein the first configuration exhibits a smaller outer diameter than said second configuration.

5. The stent of claim 1 wherein a first portion of the outer surface contacts a second portion of the exterior surface to define the elongate fold.

6. The stent of claim 1 wherein the shape-memory is a biodegradable material.

7. The stent of claim 1 wherein the polymeric material has a T_{g} selected to be greater than human body temperature.

8. The stent of claim 1 wherein the polymeric material has a T_{g} selected to be between about 35° C. and about 80° C.

9. The stent of claim 1 wherein the polymeric material has a T_{g} selected to be between about 45° C. and about 70° C.

10. The stent of claim 1 wherein the polymeric material is selected from the group consisting of: polymerized monomers derived from l, d, or d/l lactide; glycolide; ethers; ethylene; propylene; butene-1; pentene-1; hexene-1, 4-methylpentene-1; styrene; norbornene; butadiene; acrylate; methacrylate; methyl methacrylate; caprolactone; hydroxy buteric acid; hydroxy valeric acid; and mixtures of these monomeric repeating units.

11. The stent of claim 1 having a plurality of folds.

12. A vascular stent comprising a tube formed of a resorbable, shape-memory polymeric material, said tube comprising a resistive wire embedded within the shape memory polymeric material, said tube provided in a first configuration suitable for vascular implantation and deformable to a second configuration upon application of selected stimuli.

13. The stent of claim 12 wherein the polymeric material is selected from the group consisting of: polymerized monomers derived from l, d, or d/l lactide; glycolide; ethers; ethylene; propylene; butene-1; pentene-1; hexene-1, 4-methylpentene-1; styrene; norbornene; butadiene; acrylate; methacrylate; methyl methacrylate; caprolactone; hydroxy buteric acid; hydroxy valeric acid; and mixtures of these monomeric repeating units.

14. The stent of claim 12 wherein the polymeric material has a T_{g} selected to be greater than human body temperature.

15. The stent of claim 12 wherein the polymeric material has a T_{g} selected to be between about 35° C. and about 80° C.

16. The stent of claim 12 wherein the polymeric material has a T_{g} selected to be between about 45° C. and about 70° C.

17. The stent of claim 12 wherein the tube in the first configuration is elongate and defines a longitudinal direction, said tube comprising one or more folds extending along the longitudinal direction.

18. The stent of claim 12 wherein the tube includes a plurality of openings therethrough.

19. A stent comprising:
a body formed of a shape memory polymeric material, said body provided in a first configuration defining a substantially planar ribbon having an first surface and an opposite second surface; and
a resistive wire positioned proximate to the first surface, wherein said body upon application of an electrical current through said wire is deformable to a second configuration different from the first configuration.

20. The stent of claim 19 wherein the ribbon includes a mesh portion.

21. The stent of claim 19 wherein the body defines an imperforate ribbon.

22. The stent of claim 19 wherein the second configuration approximates a helix.

23. The stent of claim 19 wherein the wire is positioned adjacent the first surface.

24. The stent of claim 19 wherein the wire is positioned between the first surface and the second surface.

25. The stent of claim 19 wherein the shape memory polymeric material is a biodegradable material.

26. The stent of claim 19 wherein the polymeric material has a T_{g} selected to be greater than human body temperature.

27. The stent of claim 19 wherein the polymeric material is selected from the group consisting of: polymerized monomers derived from l, d, or d/l lactide; glycolide; ethers; ethylene; propylene; butene-1; pentene-1; hexene-1, 4-methylpentene-1; styrene; norbornene; butadiene; acrylate; methacrylate; methyl methacrylate; caprolactone; hydroxy buteric acid; hydroxy valeric acid; and mixtures of these monomeric repeating units.

28. A method of treatment comprising implanting a vascular stent into a vascular vessel or artery, said stent comprising a body formed of a shape-memory polymeric material, said body having an inner surface and an opposite exterior surface and a resistive heating element positioned proximate to the inner surface, said body provided in a first configuration having a smaller cross-sectional profile and upon application of a current to the wire, said body is deformable to a second configuration different from the first configuration.

29. The method of claim 28 wherein the body in the first configuration defines a substantially flat, elongate ribbon.

30. The stent of claim 28 wherein the body in the first configuration approximates an elongated sleeve.

31. The method of claim 30 wherein the stent in the first configuration has a smaller outer diameter than the stent in the second configuration.

32. The method of claim 28 wherein the stent deforms to the second configuration upon application of thermal energy.

33. The method of claim 28 wherein the second configuration approximates an elongate cylindrical sleeve.

34. The method of claim 28 wherein the second configuration approximates a helix or double helix.

35. The method of claim 28 wherein the shape-memory polymeric material is biodegradable.

36. The method of claim 28 wherein the wire is positioned between the inner and exterior surfaces.

37. The method of claim 28 wherein the wire is positioned adjacent the inner surface.

38. The method of claim 28 wherein the stent is implanted using a catheter.

39. The method of claim 38 wherein said application of application of a current to the wire releases the stent from the catheter.

40. The method of claim 28 wherein the polymeric material is selected from the group consisting of: polymerized monomers derived from l, d, or d/l lactide; glycolide; ethers; ethylene; propylene; butene-1; pentene-1; hexene-1, 4-methylpentene-1; styrene; norbornene; butadiene; acrylate; methacrylate; methyl methacrylate; caprolactone; hydroxy buteric acid; hydroxy valeric acid; and mixtures of these monomeric repeating units.
